(19) United States
(12) Patent Application Publication
(10) Pub. No.: US 2024/0366498 A1
(43) Pub. Date: Nov. 7, 2024

(54) Reducing the Risk of Infection from Bacteria Present in Skin Pores During Surgery

(71) Applicant: The Board of Trustees of the Leland Stanford Junior University, Stanford, CA (US)

(72) Inventors: Michael Freehill, Stanford, CA (US); Kerwyn Huang, Palo Alto, CA (US); Eric A. Appel, Palo Alto, CA (US)

(21) Appl. No.: 18/580,859

(22) PCT Filed: Jul. 29, 2022
(86) PCT No.: PCT/US2022/038934

§ 371 Date: Jan. 19, 2024

Related U.S. Application Data
(60) Provisional application No. 63/227,673, filed on Jul. 30, 2021.
(60) Provisional application No. 63/306,642, filed on Feb. 4, 2022.

(51) Int. Cl. A61K 9/00; A61K 45/06; A61P 31/02; A61P 31/04

(52) U.S. Cl. CPC A61K 9/0014; A61K 45/06; A61P 31/02; A61P 31/04

(57) ABSTRACT

Provided are methods of preparing skin for surgery to reduce a risk of infection by bacteria present in skin pores. Also provided are kits for performing such methods. The methods include exposing the bacteria by applying and then removing a first adhesive member from the skin, and then applying an antibacterial agent to the skin.

### CROSS REFERENCE TO RELATED APPLICATIONS

This application claims priority to U.S. Provisional Application 63/306,642, filed Feb. 4, 2022, and U.S. Provisional Application 63/227,673, filed Jul. 30, 2021, the disclosures of which are incorporated herein by reference.

### INTRODUCTION

Cutibacterium acnes is a lipophilic, anaerobic, Gram-positive bacterium that mainly colonizes the pilosebaceous glands of human skin. Although it is a native organism to the body, it is not meant to inhabit regions deeper into the shoulder or shoulder joint. C. acnes is the leading cause of prosthetic joint infection (PJI) after shoulder replacement, occurring in roughly 1% of patients, and can lead to implant failure within the first 2 years after surgery (Achermann et al., 2014; Dines et al., 2006; Portillo et al., 2013). Although these infection rates may appear low, positive cultures have been reported in 19% of primary shoulder replacements, with males approaching a positive culture rate of 50% (Wong et al., 2018); suggesting that many cases are undiagnosed and creating a treatment dilemma. The anaerobic and lipid-rich conditions within the pilosebaceous glands provides an optimal microenvironment for C. acnes growth, especially in cases wherein there is a blocked follicle (Brown and Shalita, 1998).

Furthermore, up to 60% of shoulders undergoing revision replacement have been reported to be culture-positive for C. acnes (Topolski et al., 2006). Shoulder arthroplasty procedures include anatomic total shoulder arthroplasty, reverse total shoulder arthroplasty, and hemiarthroplasty. The number of primary shoulder arthroplasties increased by 104% between 2011 and 2017. By 2025, a linear model predicts shoulder arthroplasty volume will increase by 67% to >174,000 annual procedures, with a Poisson model predicting a 235% increase to 350,558 procedures (Wagner et al., 2020). Of note, these growth rate projections outpace those of total hip and total knee arthroplasty (Wagner et al., 2020). Data from 2011 report median hospitalization costs for PJI of $17,163, with $16,132 for total shoulder arthroplasty, $13,955 for hemiarthroplasty, and $20,007 for reverse arthroplasty (Padegimas et al., 2015). Although hospitalization costs for PJI are comparable to those of primary arthroplasty, they are incurred after the original cost of shoulder arthroplasty and do not account for lost time from work, cost to society, and emotional well-being. Thus, strategies to prevent even low levels of PJI can have massive economic impact. Given the importance of C. acnes as a key player in the skin microbiota of the shoulder region, targeted decolonization must be carefully considered, with antibiotics or other strategies.

Up to this point more emphasis has been focused on treatment once the infection has occurred, as well as on new technologies and clinical findings for diagnosis. Previous efforts at prevention work have focused on skin preparations pre-operatively, but they do not penetrate the dermal layer fully. Although C. acnes loads can be decreased with this strategy, neutralization is clearly not complete (Chalmers et al., 2019; Duvall et al., 2020). A recent study reported that disinfection of the subcutaneous tissue with an additional preparation of povidone-iodine solution reduced the C. acnes culture rate during primary open shoulder surgery by three-fold (Moor et al., 2021). However, limitations persist as deep soft tissue cultures were not obtained and unroofed follicles on the skin likely still inoculate the wound and remain undetected. At this time, addressing these shortcomings remains a tremendous unmet need in shoulder surgery.

### SUMMARY

Provided are methods of preparing skin for surgery to reduce a risk of infection by bacteria present in skin pores. The methods include exposing the bacteria by applying and then removing a first adhesive member from the skin, and then applying an antibacterial agent to the skin. Simply applying an antibacterial agent to skin might not kill bacteria in skin pores which could be introduced into the surgical site since material on the surface of skin and in an upper region of the skin pore might block the antibacterial agent from contacting the bacteria. However, by first exposing the bacteria by removal of such material, the antibacterial agent can more successfully kill bacteria in skin pores, such as Cutibacterium acnes bacteria, reducing risk of infection during surgery.

### BRIEF DESCRIPTION OF THE DRAWINGS

FIG. 1 shows a right cylinder, wherein the first adhesive member can have the shape of the curved surface of the right cylinder.

FIG. 2 shows a cylinder with two rounded ends, wherein the first adhesive member can have the shape of the curved surface of the cylinder.

FIG. 3 shows a roller having a first adhesive member with rounded ends and a central member. The section of the central member that is not covered by the first adhesive member can be used as a handle that is gripped by a user.

FIG. 4 shows an example of how optical density (OD) data was recorded over about 70 hours after hydrogen peroxide was administered to different strains of C. acnes.

FIG. 5 shows how IC₅₀ was determined from a comparison of OD at different concentrations.

FIG. 6 shows a calculation where IC₅₀ fold changes were calculated. The IC₅₀ fold change was calculated from the matrix of data at the top: for each column, the lowest IC₅₀ was normalized to 1, and then all values in that column were normalized to the lowest IC₅₀ value.

FIG. 7 shows the toxicity of antibacterial agents to human embryonic kidney (HEK-293T) cells in vitro.

FIG. 8 shows an applicator including an absorbent material and the antibacterial agent located in a reservoir.

FIG. 9 shows a first adhesive member with a concave roller and a rotational axis that is perpendicular to the handle.

FIG. 10 shows an adhesive member with a concave roller and a rotational axis that is parallel to the handle.

### DETAILED DESCRIPTION

Provided are methods of preparing skin for surgery to reduce a risk of infection by bacteria present in skin pores. Also provided are kits for performing such methods. The methods include exposing the bacteria by applying and then removing a first adhesive member from the skin, and then applying an antibacterial agent to the skin. Simply applying an antibacterial agent to skin might not kill bacteria in skin pores which could be introduced into the surgical site since material on the surface of skin and in an upper region of the skin pore might block the antibacterial agent from contacting the bacteria. However, by first exposing the bacteria by removal of such material, the antibacterial agent can more successfully kill bacteria in skin pores, such as Cutibacterium acnes bacteria, reducing risk of infection during surgery.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

Provided are methods of preparing skin for surgery to reduce a risk of infection by bacteria present in skin pores. In some cases, the methods include applying and then removing a first adhesive member from the skin, thereby exposing bacteria present in skin pores of the skin, and then applying an antibiotic to the skin. In some cases, methods include exposing the bacteria by applying and then removing a first adhesive member from the skin, and then applying a second adhesive member comprising an antibacterial agent to the skin. The second adhesive member is for introducing the antibacterial agent to the skin.

### First Adhesive Member

The exposing step can expose the bacteria by removing material from the skin surface or within the skin pore, e.g. an upper region of the skin pore. Exemplary materials include sebum, which is secreted by sebaceous glands in a skin pore, dead skin cells, hair follicles, and a non-biological material such as dirt. After the exposing, the antibacterial agent can more easily penetrate into the skin pores, thereby contacting and killing or inhibiting the exposure and possible introduction of the bacteria into the surgical site.

In some cases, the first adhesive member has a rectangular, e.g. a square, cross-section. For instance, the first adhesive member can be a pad. The first adhesive member can comprise a substrate and an adhesive. For instance, the substrate can have a first planar surface and an adhesive located on the first planar surface. In some embodiments, first planar surface has a surface area ranging from 10 cm² to 2000 cm², such as from 100 cm² to 1000 cm². In some cases the first adhesive member has a thickness, i.e. a dimension perpendicular to the first planar surface, ranging from 0.1 cm to 5 cm, such as from 0.2 cm 3 cm.

In other embodiments, the first adhesive member can have a shape that is related to the geometric shape of a cylinder. In particular cases, the first adhesive member can have the shape of the third surface of a cylinder.

As used herein, the term “cylinder” refers to a 3-dimensional object that has two parallel circular surfaces that are connected by a third surface, which is curved. The terms “third surface” of a cylinder and “curved surface” of a cylinder are used interchangeably herein. The longitudinal axis of the cylinder is an axis that is perpendicular to the circular surfaces. In some cases the longitudinal axis intersects both the center of the first circular surface and the center of the second circular surface. A transverse axis of the cylinder is an axis that intersects the longitudinal axis and is perpendicular to the longitudinal axis. A cylinder also has multiple cross-sections, each of which are perpendicular to the longitudinal axis.

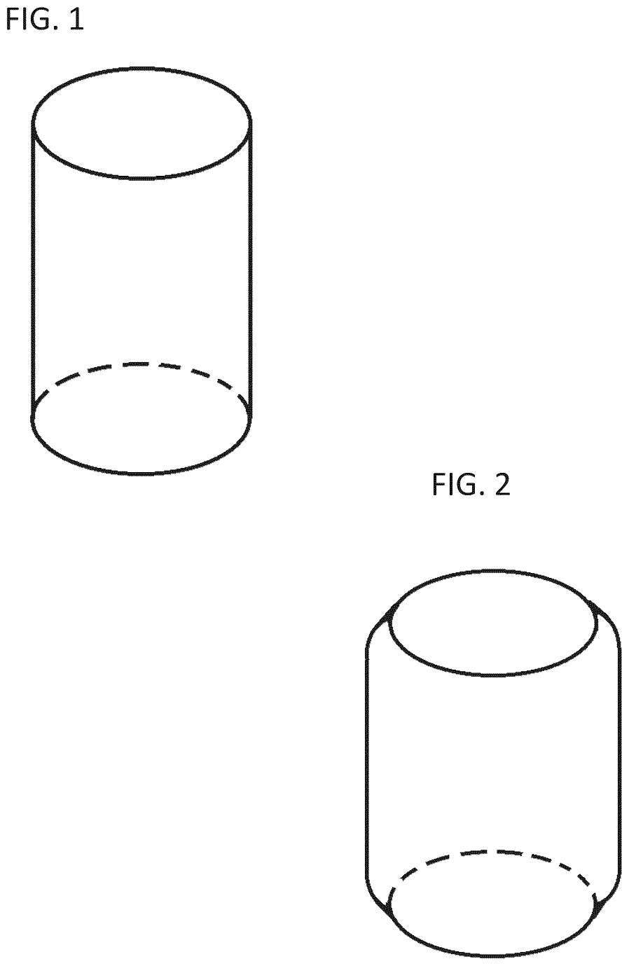

In some cases, the cylinder is a “right” cylinder, which is when the third surface forms a 90° (i.e. right) angle with the circular ends. FIG. 1 shows an example of a right cylinder.

In some cases, the third surface of the cylinder is said to be “straight”. FIG. 1 shows an example of a cylinder with a “straight” third surface. As used herein, a straight third surface means that each of the cross-sections of the third surface are circles, and each of such cross-sectional circles have the same radius. It will be understood that imperfections in manufacturing will result in unavoidable deviations from perfect circularity and unavoidable differences in the radius of each cross-section. Hence, the term “same radius” means that the largest radius is 110% or less of the smallest radius, such as 105% or less, 102% or less, or 101% or less. Similarly, when assessing how close a single cross-section is to a perfect circle, such a cross-section can be considered circular if the distance from the center to furthest point is 110% or less of the distance from the center to the closest point, such as 105% or less, 102% or less, or 101% or less.

In some cases, the third surface of the cylinder is not “straight”. For instance, each cross section of the cylinder might be circular, but at least one cross-sections might have a different radius than another cross-section. FIG. 2 shows an embodiment of such a non-straight cylinder wherein each cross-section is circular, but the cross-sections at each end are narrower than the cross-sections in the middle, i.e. they have smaller radii. Such an embodiment can be said to have rounded ends and can be considered a “convex” shape. In some cases, a cylinder can have a diameter that varies along the longitudinal axis, i.e. each cross-section is circular but there is variability in the radii of the cross-sections. In some cases the cylinder has one rounded end. In some cases the cylinder has two rounded ends.

In some cases, there can be a “concave shape”. In other words, there can be a non-straight cylinder wherein each cross-section is circular, but the cross-sections at each end are larger than the cross-sections in the middle, i.e. they have larger radii.

In some embodiments the first adhesive member can have a convex shape, i.e, wherein the circular cross-sections at each end of the cylinder have a smaller radii than a cross-section in the middle of the cylinder. In some embodiments the first adhesive member can have a concave shape, i.e, wherein the circular cross-sections at each end of the cylinder have a larger radii than a cross-section in the middle of the cylinder.

Hence, the first adhesive member can have the shape of the third surface of a cylinder, e.g. a right cylinder, a straight cylinder, or a non-straight cylinder. For instance, the first adhesive member can comprise a substrate and an adhesive. The substrate can have the shape of the third surface of a cylinder, and the adhesive can be in contact with the outer surface of the substrate. In some cases the substrate also includes an inner surface, i.e. it is hollow. In other cases the substrate does not include an inner surface.

In some cases the length of the cylinder ranges from 2 cm to 20 cm, such as from 4 cm to 15 cm, wherein the length is the distance along the longitudinal axis between the circular surfaces. In some cases the average radius of the cylinder's cross-sections ranges from 0.5 cm to 7 cm, such as from 1 cm to 5 cm.

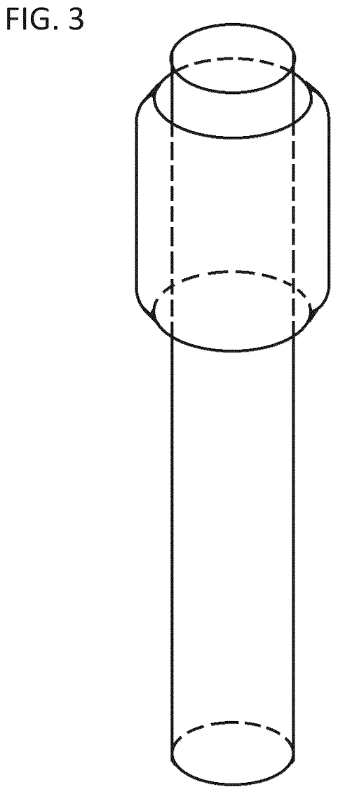

In such embodiments the method can involve a roller, wherein the roller comprises a cylindrical first adhesive member and a central member. The applying and removing can comprise moving the roller across the skin such that the first adhesive member rotates around the central member. As such, each location of the first adhesive member is first contacted with and then pulled away from the skin, thereby performing the applying and removing. This motion can be performed in a similar manner to how a lint roller is used to remove lint from an article of clothing, such as a cotton shirt. In some embodiments, the length of the central member will be greater than the length of the first adhesive member in order to provide a handle for a user to grip. Thus, the length of the central member can be 5 cm to 30 cm greater than the length of the first adhesive member. When the central member forms the handle, the axis of the handle is parallel to the axis of rotation of the roller, e.g. as shown in FIG. 3. In other cases, the device includes other members such that a handle is non-parallel. For example, the handle can be perpendicular to the axis of the central member and the rotation of the roller, e.g. as shown in FIG. 9.

In some cases, it can be difficult to move the roller across the skin while also retaining constant contact between the roller and skin. In some cases a first adhesive member with a variable radius, such as having rounded ends in a convex fashion, can allow the roller to move more easily across an uneven skin surface while retaining contact between the roller and skin. A concave arrangement can also help the roller move across the skin.

FIG. 3 shows a roller having a first adhesive member with rounded ends and a central member. The first adhesive member has a circular cross section and is configured to rotate freely around the central member. The section of the central member that is not covered by the first adhesive member can function as a handle and be gripped by the hand of a user.

### Antibacterial Agent

As described above, the method also involves applying an antibacterial agent to the skin. In some cases, the antibacterial agent is located in a second adhesive member that is adhered to the skin. In some cases the second adhesive member is allowed to remain adhered, e.g. and the method further comprises performing a surgical incision into the skin by cutting through the second adhesive member. Since the skin remains covered until the moment of incision, the risk and presence of bacteria at the incision site can be reduced.

In some cases, the antibacterial agent is located in an absorbent material, wherein applying the antibacterial agent to the skin comprises contacting the absorbent material to the skin. In some cases, the absorbent material is a solid surface onto which the antibacterial agent can adhere. In other cases, the absorbent material is a porous material, such as a sponge, that can absorb a relatively high amount of antibacterial agent due to its high surface area and shape. After contacting, the absorbent material can be moved away from the skin.

In some cases the method involves an applicator comprising an absorbent material and the antibacterial agent located in a reservoir, where applying the antibacterial agent includes releasing the antibacterial agent from the reservoir and into the absorbent material and thereafter contacting the absorbent material to the skin. In some of such cases, the amount of antibacterial agent can be predetermined in order to deliver a desired amount of antibacterial agent.

In some cases the absorbent material has a rectangular, square, circular, or oval cross-section. In some of such cases the antibacterial agent can be applied to the skin in a manner similar to how paint is applied with a paintbrush. Such shapes can be used regardless of whether the antibacterial agent was previously placed into contact with the absorbent material, or whether it was placed into a reservoir and then released into the absorbent material, as described above.

In some cases the absorbent material has a circular cross-section. In some cases, the absorbent material has the shape of a curved surface of a right cylinder, a straight cylinder, or a non-straight cylinder. In some instances the method involves an applicator that is a second roller that further comprises a second central member, wherein applying the antibacterial agent comprises moving the second roller across the skin such that the absorbent material rotates around the second central member while the absorbent material contacts the skin. FIG. 3 was previously described as showing an exemplary roller with a central member and a first adhesive member. However, the shape of the element in FIG. 3 can also be used as a second roller comprising a second central member and an absorbent material with a circular cross-section.

In fact, the absorbent material can have any of the shapes previously described for the first adhesive member. The absorbent material can be straight cylinder, a non-straight cylinder, a convex shape, a concave shape, or any other suitable shape.

In some embodiments the absorbent material can have a convex shape, i.e, wherein the circular cross-sections at each end of the cylinder have a smaller radii than a cross-section in the middle of the cylinder. In some embodiments the absorbent material can have a concave shape, i.e, wherein the circular cross-sections at each end of the cylinder have a larger radii than a cross-section in the middle of the cylinder.

### Location of Skin

The present methods can be used to prepare any section of skin, e.g. because bacteria such as C. acnes can be present on any part of the skin. In some cases, the skin is located at the surgical site. In other cases, the skin is located near the surgical site, e.g. because contact between objects in a surgical setting could transfer bacteria from a nearby skin location to the location of a surgical incision.

In some instances, the skin is located at the shoulder. For instance, the skin can cover a clavicle bone, a scapula bone, an upper half of a humerus bone, or a combination thereof. Preparation of such skin locations can be performed during shoulder surgery. In some cases, the skin is located at the posterior thorax. For instance, the skin can cover a bone of the vertebral column, which is also referred to herein as the backbone or the spine. In humans the vertebral column includes cervical vertebrae, thoracic vertebrae, lumbar vertebrae, the sacrum, and the coccyx. In some instances, the skin covers one or more of such vertebrae. As such, the methods can be used to prepare skin for spinal surgery. In some embodiments, the skin location be at both the shoulder and the vertebral column. For instance, the prepared skin location can be large enough that it covers both a scapula bone and a thoracic vertebrae.

As used herein, the expression “skin covers a particular bone” means that if a line is drawn perpendicular to the plane of the skin location, then the line will intersect the particular bone.

In some cases, the skin is located on the posterior side of the subject, e.g. the posterior thorax. The skin can also be located at the anterior thorax, a leg, an arm, the hips, the neck, the head, or a combination thereof.

### Kits

Provided are kits for preparing skin for surgery to reduce a risk of infection by bacteria present in skin pores. In some cases, the kits include a first adhesive member configured to be applied and then removed from the skin, thereby exposing bacteria present in skin pores of the skin, along with an antibacterial agent.

In some cases the kits include one or more of the elements described as being used in the discussion of methods, above.

In some embodiments, the kits include a roller that comprises the first adhesive member and a central member, wherein the first adhesive member can rotate around the central member. As used herein, the terms “rotate” and “freely rotate” as used interchangeably to indicate that a first element (e.g. the first adhesive member) can move around an axis regardless of the orientation of a second element (e.g. the central member). In contrast, restricted rotation means that a first element can only rotate a certain amount, e.g. less than 5°, unless the second element also rotates in the same direction.

In some embodiments, the kit includes an applicator comprising an absorbent material and a reservoir configured to contain the antibacterial agent, where the applicator is configured such that antibacterial agent inside the reservoir can be released into the absorbent material. In some kits the antibacterial agent is already located in the reservoir, and in some kits the antibacterial agent is located separately (e.g. in a separate package or container) and outside the reservoir.

In some cases, kits include a first adhesive member configured to expose bacteria present in the skin pores after applying and then removing the first adhesive member from the skin; and a second adhesive member that includes an antibacterial agent. The antibacterial agent can be retained on the skin throughout the surgery and an incision and the surgery can be carried out through the second adhesive member.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Effect of Antibacterial Agents on C. Acnes

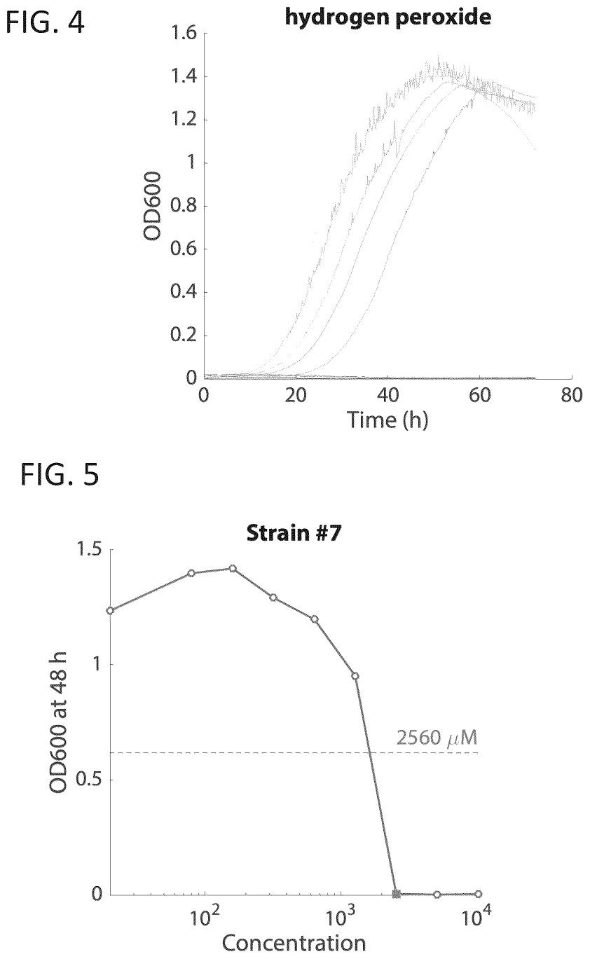

Seven different strains of Cutibacterium acnes were grown in test tubes in Reinforced Clostridial Medium (RCM). Each strain was divided into different wells of mulita-well plates (96-well and 384-well plates were used). 80 μl was used and a 1:200 dilution from saturated cultures. Either an antibacterial agent or vehicle (as a control) was added to each well. Absorbance was measured and quantified as optical density (OD) as a proxy for cell biomass. The OD was recorded at regular intervals over several hours. For example, FIG. 4 shows an example of OD measurements over about 70 hours for different strains that had been administered hydrogen peroxide. The OD at 48 hours was estimated by calculating the average OD between 47 and 49 hours, and this value was used for further analyses. OD600 is the OD at 600 nm. The OD is listed in arbitrary units determined by the spectrophotometers.

Such OD data was collected for different concentrations of each antibacterial agent. For example, FIG. 5 shows the OD at 48 hours when strain 7 of c. acnes was administered different concentrations. The horizontal axis has units of μM (micromoles per liter). Next, IC₅₀ values were calculated by assessing when the OD value was about 0.5. For example, in FIG. 5 the IC₅₀ concentration was about 2560 UM since the experimental data has an OD of about 0.5 at that concentration.

The strains of C. acne used are shown in the table below.

| Strain | Cutibacterium acnes |
|---|---|
| 2 | BWH-002 25541381 |
| 3 | BWH-003 25505801 |
| 10 | HM-523 (HL053PA2) |
| 12 | HM-539 (HL086PA1) |
| 13 | HM-552 (HL110PA1) |
| 15 | KPL2008 |
| 16 | KPL1847 |

The IC₅₀ values in μM for different antibacterial agents in correspondence with different strains is shown in the tables below.

| Strain | Amoxicillin | Ceftriaxone | Cefepime | Ertapenem |
|---|---|---|---|---|
| 2 | 0.078 | 0.0097 | 0.31 | 0.039 |
| 3 | 0.0098 | 0.0098 | 2.5 | 0.16 |
| 10 | 0.078 | 0.0098 | 0.31 | 0.078 |
| 12 | 0.039 | 0.0098 | 0.16 | 0.0098 |
| 13 | 0.019 | 0.0098 | 0.31 | 0.039 |
| 15 | 0.16 | 0.08 | 2.5 | 0.16 |
| 16 | 0.31 | 0.078 | 2.5 | 0.31 |

| Strain | Vancomycin | Hydrogen Peroxide | Benzoyl Peroxide |
|---|---|---|---|
| 2 | 0.16 | 1280 | 2560 |
| 3 | 0.15 | 2560 | >2560 |
| 10 | 0.16 | 2560 | 1280 |
| 12 | 0.31 | 1280 | 2560 |
| 13 | 0.16 | 640 | 640 |
| 15 | 0.31 | 2560 | 640 |
| 16 | 0.31 | 2560 | 640 |

| Strain | Ciprofloxacin | Metronidazole | Kanamycin |
|---|---|---|---|
| 2 | 1.25 | >5120 | 640 |
| 3 | 5 | >5120 | 80 |
| 10 | 0.31 | >5120 | 320 |
| 12 | 1.25 | 5120 | 160 |
| 13 | 1.25 | 5120 | 320 |
| 15 | 5 | 5120 | 640 |
| 16 | 5 | >5120 | 160 |

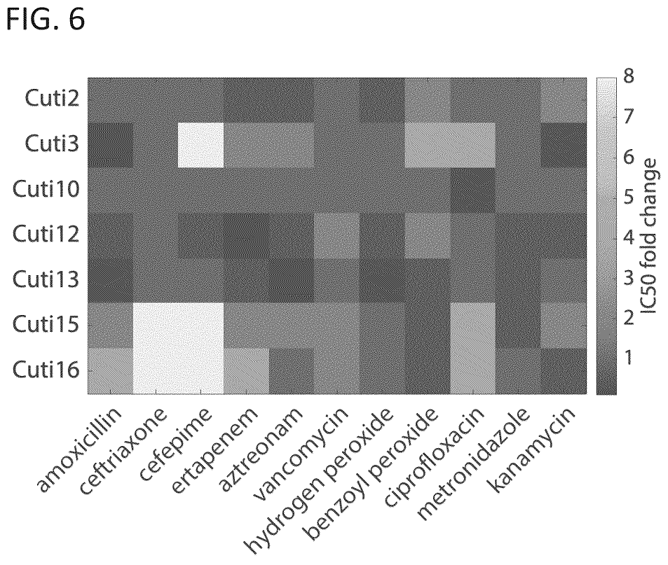

Such data indicated that certain antibacterial agents had relatively low IC₅₀ values, such as amoxicillin and ceftriaxone. In addition, FIG. 6 shows a calculation wherein IC₅₀ fold changes were calculated. The IC₅₀ fold change was calculated from the matrix of data at the top: for each column, the lowest IC₅₀ was normalized to 1, and then all values in that column were normalized to the lowest IC₅₀ value.

### Example 2: Toxicity of Antibacterial Agents to Human Cells In Vitro

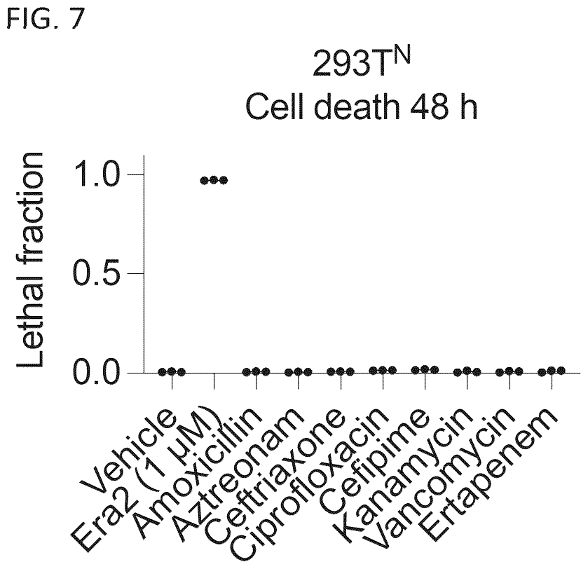

In addition, the toxicity of such antibacterial agents to human cells was tested. In particular, they were tested against human embryonic kidney (HEK-293T) cells, which are typical cell lines used for drug profiling. Compounds were tested across a dose-response series and over time. Across three replicates, none of them were lethal (i.e. toxic) at the highest dose they could test (250 μM) in HEK-293T cells. None of the antibiotics elevated the lethal fraction off of zero, as shown in FIG. 7. A compound that causes lethality, Era2, was included as a positive control for the assay.

### Example 3: Testing Adhesive Members

Studies were conducted to determine the relative advantages and disadvantages of different adhesive members at exposing bacteria present in skin pores of the skin. Several commercially sold adhesive members were tested for their interaction with skin. It was determined that some adhesive members appeared to have disadvantageously low adhesiveness, and therefore might not sufficiently expose the skin pores. In contrast, other adhesive members had adhesiveness that was potentially too high or unnecessarily high and might cause difficulty in use. It was found that the MR.SIGAR lint roller and the Polardo® lint roller appeared to have a potentially advantageously medium level of adhesiveness. Such lint rollers had a handle shaped and dimensioned for grip by the human hand and a cylindrical adhesive member that rotated freely around the handle.

### Example 4: Applicator for Antibiotic Agent

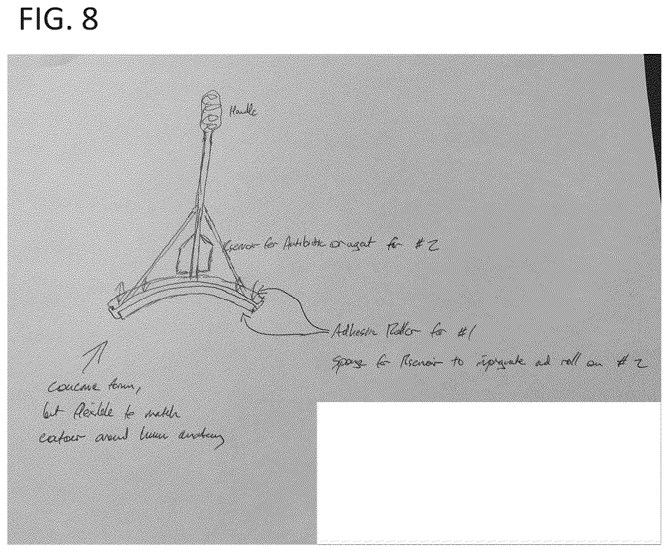

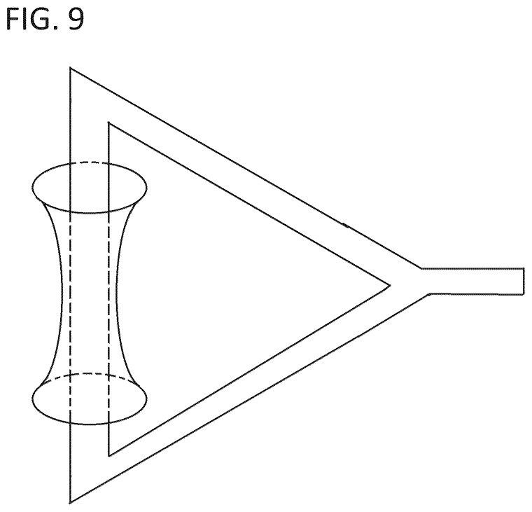

FIG. 8 shows a device including a reservoir and a roller. The text at the bottom left reads “concave form, but flexible to match contour around human anatomy”. The text at the right reads “Adhesive roller for #1 sponge for reservoir to impregnate and roll on #2”. The middle text reads “Reservoir for Antibiotic agent for #2”. The top text reads “handle”. The device can be used to apply an antibacterial agent to the skin. For example, the antibacterial agent can be located in the central reservoir, and then released from the reservoir to be absorbed into the absorbent material of the roller, and then applied to the skin. The FIG. 8 device has a roller with a concave shape in the longitudinal direction (e.g., curved along the longitudinal axis of the roller) that can advantageously make it easier to apply the antibacterial agent to the skin while accommodating for the curves and contours of uneven parts of the skin, e.g. around the curvature of the shoulder.

### Example 5: Adhesive Member

The first adhesive member which exposes bacteria present in skin pores can also have a concave configuration in a manner similar to the FIG. 8 device for applying the antibacterial agent. In particular, the adhesive member can be a roller that has a concave shape. FIG. 9 shows an embodiment of an adhesive member with a concave shape that is attached to a structure having a handle. This member can be pushed along the skin.

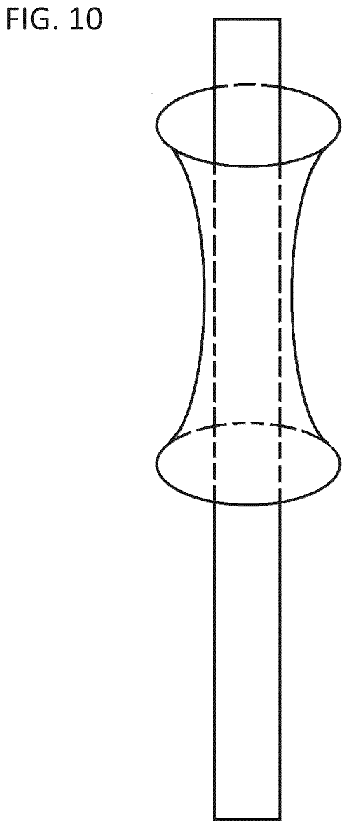

FIG. 10 shows an alternative first adhesive member with that is similar to FIG. 3, except that the adhesive member section is concave in FIG. 10 whereas it is convex in FIG. 3.

### Embodiments

Notwithstanding the appended claims, the disclosure is also defined by the following clauses:
1. A method of preparing skin for surgery to reduce a risk of infection by bacteria, the method comprising:
   applying and then removing a first adhesive member from the skin, thereby exposing bacteria present in skin pores of the skin; and applying an antibacterial agent to the skin.
2. The method of clause 1, wherein the first adhesive member has a rectangular or square cross-section.
3. The method of clause 1, wherein the first adhesive member has a circular cross-section.
4. The method of clause 3, wherein the first adhesive member has a convex shape or a concave shape.
5. The method of clause 4, wherein the first adhesive member has the shape of the curved surface of a cylinder with one or more rounded ends.
6. The method of any one of clauses 4-6, wherein a roller comprises the first adhesive member and a central member, wherein the applying and then removing comprises moving the roller across the skin such that the first adhesive member rotates around the central member.
7. The method of any one of clauses 1-6, wherein the antibacterial agent is located in a second adhesive member, wherein applying the antibacterial agent to the skin comprises adhering the second adhesive member to the skin.
8. The method of clause 7, further comprising performing a surgical incision into the skin by cutting through the second adhesive member.
9. The method of any one of clauses 1-8, wherein the antibacterial agent is located in an absorbent material, wherein applying the antibacterial agent to the skin comprises contacting the absorbent material with the skin.
10. The method of clauses 1-8, wherein an applicator comprises an absorbent material and the antibacterial agent located in a reservoir, wherein applying the antibacterial agent comprises releasing the antibacterial agent from the reservoir and into the absorbent material and thereafter contacting the absorbent material with the skin.
11. The method of clause 10, wherein the absorbent material has a rectangular, square, or oval cross-section.
12. The method of any one of clauses 10-11, wherein the applicator further comprises a handle, wherein the applicator is configured such that the absorbent material cannot rotate relative to the handle.
13. The method of any one of clauses 9-12, wherein the absorbent material has a circular cross-section.
14. The method of clause 13, wherein the absorbent material has the shape of the curved surface of a right cylinder.
15. The method of clause 13, wherein the absorbent material a convex shape or a concave shape.
16. The method of any one of clauses 10-15, wherein the applicator is a second roller that further comprises a second central member, wherein applying the antibacterial agent comprises moving the second roller across the skin such that the absorbent material rotates around the second central member.
17. The method of any one of clauses 1-16, wherein the skin pores each comprise a hair follicle.
18. The method of any one of clauses 1-16, wherein the skin pores do not comprise a hair follicle.
19. The method of any one of clauses 1-18, wherein the applying and removing removes bacteria from a surface of the skin, exposes bacteria present on the surface of the skin, or a combination thereof.
20. The method of any one of clauses 1-19, wherein the antibacterial agent is effective against a bacteria present in a hospital.
21. The method of any one of clauses 1-20, wherein the antibacterial agent is effective against Cutibacterium acnes bacteria.
22. The method of any one of clauses 1-21, wherein the antibacterial agent is an antiseptic.
23. The method of any one of clauses 1-21, wherein the antibacterial agent is an antibiotic.
24. The method of any one of clauses 1-21, wherein the antibacterial agent is selected from the group consisting of: chlorhexidine, triclosan, penicillin, iodine, providone-iodine, hydrogen peroxide, benzoyl peroxide, ethanol, isopropanol, an alkyldimethylbenzyl ammonium chloride, penicillin, amoxicillin, ceftriaxone, cefipime, ertapenem, aztreonam, and vancomycin.
25. The method of any one of clauses 1-24, wherein the applying the second adhesive member is performed with a concentration of the antibacterial agent and for a time period sufficient to effectively kill the bacteria while also being safe to the skin.
26. The method of clause 25, wherein the concentration and time were determined by in vitro testing of the antibacterial agent against skin cells.
27. The method of any one of clause 1-26, wherein the skin is located at or near a surgical site.
28. The method of any one of clause 1-27, wherein the skin is located at a surgical incision site.
29. The method of any one of clauses 1-28, wherein the skin is located at the shoulder.
30. The method of clause 29, wherein the skin covers a clavicle bone, a scapula bone, an upper half of a humerus bone, or a combination thereof.
31. The method any one of clauses 1-30, wherein the skin is located at the posterior thorax.
32. The method of clause 31, wherein the skin covers a bone of the vertebral column.
33. A kit for preparing skin for surgery to reduce a risk of infection by bacteria, the kit comprising:
   a first adhesive member configured to be applied and then removed from the skin, thereby exposing bacteria present in skin pores of the skin; and
   an antibacterial agent.
34. The kit of clause 33, wherein the first adhesive member has a rectangular or square cross-section.
35. The kit of clause 33, wherein the first adhesive member has a circular cross-section.
36. The kit of clause 35, wherein the first adhesive member has the shape of the curved surface of a right cylinder.
37. The kit of claim 36, wherein the first adhesive member has a convex shape or a concave shape.
38. The kit of any one of claims 35-37, wherein a roller comprises the first adhesive member and a central member, wherein the first adhesive member can rotate around the central member.
39. The kit of any one of claims 33-38, wherein the antibacterial agent is located in a second adhesive member.
40. The kit of claim 39, wherein the second adhesive member is configured to be cuttable by a scalpel.
41. The kit of any one of claims 33-40, further comprising an absorbent material comprising the antibacterial agent.
42. The kit of any one of claims 33-40, further comprising an applicator comprising an absorbent material and a reservoir configured to contain the antibacterial agent, wherein the applicator is configured such that antibacterial agent inside the reservoir can be released into the absorbent material.
43. The kit of any one of claims 41-42, wherein the absorbent material has a rectangular, square, or oval cross-section.
44. The kit of claim 43, wherein the applicator further comprises a handle, wherein the applicator is configured such that the absorbent material cannot rotate relative to the handle.
45. The kit of any one of claims 41-42, wherein the absorbent material has a circular cross-section.
46. The kit of claim 45, wherein the absorbent material has the shape of the curved surface of a right cylinder.
47. The kit of claim 45, wherein the absorbent material has a convex shape or a concave shape.
48. The kit of any one of clauses 45-47, wherein the applicator is a second roller that further comprises a second central member, wherein the absorbent material can rotate around the second central member.
49. The kit of any one of clauses 33-48, wherein the antibacterial agent is effective against a bacteria present in a hospital.
50. The kit of any one of clauses 33-48, wherein the antibacterial agent is effective against Cutibacterium acnes bacteria.
51. The kit of any one of clauses 33-50, wherein the antibacterial agent is an antiseptic.
52. The kit of any one of clauses 33-50, wherein the antibacterial agent is an antibiotic.
53. The kit of any one of clauses 33-50, wherein the antibacterial agent is selected from the group consisting of: chlorhexidine, triclosan, penicillin, iodine, providone-iodine, hydrogen peroxide, benzoyl peroxide, ethanol, isopropanol, an alkyldimethylbenzyl ammonium chloride, penicillin, amoxicillin, ceftriaxone, cefipime, ertapenem, aztreonam, and vancomycin.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

What is claimed is:

1. A method of preparing skin for surgery to reduce a risk of infection by bacteria, the method comprising:
applying and then removing a first adhesive member from the skin, thereby exposing bacteria present in skin pores of the skin; and
applying an antibacterial agent to the skin.

2. The method of claim 1, wherein the first adhesive member has a rectangular or square cross-section.

3. The method of claim 1, wherein the first adhesive member has a circular cross-section.

4. The method of claim 3, wherein the first adhesive member has a convex shape or a concave shape.

5. The method of claim 4, wherein the first adhesive member has the shape of the curved surface of a cylinder with one or more rounded ends.

6. The method of any one of claims 4-6, wherein a roller comprises the first adhesive member and a central member, wherein the applying and then removing comprises moving the roller across the skin such that the first adhesive member rotates around the central member.

7. The method of any one of claims 1-6, wherein the antibacterial agent is located in a second adhesive member, wherein applying the antibacterial agent to the skin comprises adhering the second adhesive member to the skin.

8. The method of claim 7, further comprising performing a surgical incision into the skin by cutting through the second adhesive member.

9. The method of any one of claims 1-8, wherein the antibacterial agent is located in an absorbent material, wherein applying the antibacterial agent to the skin comprises contacting the absorbent material with the skin.

10. The method of claims 1-8, wherein an applicator comprises an absorbent material and the antibacterial agent located in a reservoir, wherein applying the antibacterial agent comprises releasing the antibacterial agent from the reservoir and into the absorbent material and thereafter contacting the absorbent material with the skin.

11. The method of claim 10, wherein the absorbent material has a rectangular, square, or oval cross-section.

12. The method of any one of claims 10-11, wherein the applicator further comprises a handle, wherein the applicator is configured such that the absorbent material cannot rotate relative to the handle.

13. The method of any one of claims 9-12, wherein the absorbent material has a circular cross-section.

14. The method of claim 13, wherein the absorbent material has the shape of the curved surface of a right cylinder.

15. The method of claim 13, wherein the absorbent material a convex shape or a concave shape.

16. The method of any one of claims 10-15, wherein the applicator is a second roller that further comprises a second central member, wherein applying the antibacterial agent comprises moving the second roller across the skin such that the absorbent material rotates around the second central member.

17. The method of any one of claims 1-16, wherein the skin pores each comprise a hair follicle.

18. The method of any one of claims 1-16, wherein the skin pores do not comprise a hair follicle.

19. The method of any one of claims 1-18, wherein the applying and removing removes bacteria from a surface of the skin, exposes bacteria present on the surface of the skin, or a combination thereof.

20. The method of any one of claims 1-19, wherein the antibacterial agent is effective against a bacteria present in a hospital.

21. The method of any one of claims 1-20, wherein the antibacterial agent is effective against Cutibacterium acnes bacteria.

22. The method of any one of claims 1-21, wherein the antibacterial agent is an antiseptic.

23. The method of any one of claims 1-21, wherein the antibacterial agent is an antibiotic.

24. The method of any one of claims 1-21, wherein the antibacterial agent is selected from the group consisting of: chlorhexidine, triclosan, penicillin, iodine, providone-iodine, hydrogen peroxide, benzoyl peroxide, ethanol, isopropanol, an alkyldimethylbenzyl ammonium chloride, penicillin, amoxicillin, ceftriaxone, cefipime, ertapenem, aztreonam, and vancomycin.

25. The method of any one of claims 1-24, wherein the applying the second adhesive member is performed with a concentration of the antibacterial agent and for a time period sufficient to effectively kill the bacteria while also being safe to the skin.

26. The method of claim 25, wherein the concentration and time were determined by in vitro testing of the antibacterial agent against skin cells.

27. The method of any one of claim 1-26, wherein the skin is located at or near a surgical site.

28. The method of any one of claim 1-27, wherein the skin is located at a surgical incision site.

29. The method of any one of claims 1-28, wherein the skin is located at the shoulder.

30. The method of claim 29, wherein the skin covers a clavicle bone, a scapula bone, an upper half of a humerus bone, or a combination thereof.

31. The method any one of claims 1-30, wherein the skin is located at the posterior thorax.

32. The method of claim 31, wherein the skin covers a bone of the vertebral column.

33. A kit for preparing skin for surgery to reduce a risk of infection by bacteria, the kit comprising:
a first adhesive member configured to be applied and then removed from the skin, thereby exposing bacteria present in skin pores of the skin; and
an antibacterial agent.

34. The kit of claim 33, wherein the first adhesive member has a rectangular or square cross-section.

35. The kit of claim 33, wherein the first adhesive member has a circular cross-section.

36. The kit of claim 35, wherein the first adhesive member has the shape of the curved surface of a right cylinder.

37. The kit of claim 36, wherein the first adhesive member has a convex shape or a concave shape.

38. The kit of any one of claims 35-37, wherein a roller comprises the first adhesive member and a central member, wherein the first adhesive member can rotate around the central member.

39. The kit of any one of claims 33-38, wherein the antibacterial agent is located in a second adhesive member.

40. The kit of claim 39, wherein the second adhesive member is configured to be cuttable by a scalpel.

41. The kit of any one of claims 33-40, further comprising an absorbent material comprising the antibacterial agent.

42. The kit of any one of claims 33-40, further comprising an applicator comprising an absorbent material and a reservoir configured to contain the antibacterial agent, wherein the applicator is configured such that antibacterial agent inside the reservoir can be released into the absorbent material.

43. The kit of any one of claims 41-42, wherein the absorbent material has a rectangular, square, or oval cross-section.

44. The kit of claim 43, wherein the applicator further comprises a handle, wherein the applicator is configured such that the absorbent material cannot rotate relative to the handle.

45. The kit of any one of claims 41-42, wherein the absorbent material has a circular cross-section.

46. The kit of claim 45, wherein the absorbent material has the shape of the curved surface of a right cylinder.

47. The kit of claim 45, wherein the absorbent material has a convex shape or a concave shape.

48. The kit of any one of claims 45-47, wherein the applicator is a second roller that further comprises a second central member, wherein the absorbent material can rotate around the second central member.

49. The kit of any one of claims 33-48, wherein the antibacterial agent is effective against a bacteria present in a hospital.

50. The kit of any one of claims 33-48, wherein the antibacterial agent is effective against Cutibacterium acnes bacteria.

51. The kit of any one of claims 33-50, wherein the antibacterial agent is an antiseptic.

52. The kit of any one of claims 33-50, wherein the antibacterial agent is an antibiotic.

53. The kit of any one of claims 33-50, wherein the antibacterial agent is selected from the group consisting of: chlorhexidine, triclosan, penicillin, iodine, providone-iodine, hydrogen peroxide, benzoyl peroxide, ethanol, isopropanol, an alkyldimethylbenzyl ammonium chloride, penicillin, amoxicillin, ceftriaxone, cefipime, ertapenem, aztreonam, and vancomycin.
